(19) United States
(12) Patent Application Publication
(10) Pub. No.: US 2026/0085333 A1
(43) Pub. Date: Mar. 26, 2026

(54) BIOCATALYTIC METHOD FOR ENHANCING ARSENIC METHYLATION AND VOLATILIZATION OF CLOSTRIDIUM AND USE THEREOF

(71) Applicant: Institute of Eco-environmental and Soil Sciences, Guangdong Academy of Sciences, Guangdong (CN)

(72) Inventors: Liping FANG, Guangdong (CN); Guanhong CHEN, Guangdong (CN); Fangbai LI, Guangdong (CN); Yongmei LIANG, Guangdong (CN)

(21) Appl. No.: 19/312,850

(22) Filed: Aug. 28, 2025

(30) Foreign Application Priority Data
Sep. 24, 2024 (CN) 202411328846.8

(51) Int. Cl. C12P 3/00

(52) U.S. Cl. CPC C12P 3/00

(57) ABSTRACT

The present disclosure belongs to the field of microbial technology, and discloses a biocatalytic method for enhancing arsenic methylation and volatilization capabilities of Clostridium and a use thereof. Specifically, the present disclosure discloses use of resazurin in enhancing arsenic methylation capability of bacteria. The present disclosure proposes for the first time that resazurin may promote the functions of bacteria (e.g. Clostridium sporogenes) in production and volatilization, as well as cell adsorption of methyl arsenic. Experimental results demonstrate that exogenous addition of resazurin may significantly improve the methylation and volatilization effects of inorganic trivalent arsenic by Clostridium sporogenes, providing an effective means for the remediation of arsenic-contaminated soil and water bodies.

### CROSS-REFERENCE TO RELATED APPLICATION

The present application is based on and claims the benefit of priority of Chinese Patent Application No. 202411328846.8 filed on Sep. 24, 2024, the entirety of which is incorporated by reference herein.

### TECHNICAL FIELD

The present disclosure belongs to the field of microbial technology, and in particular, relates to a biocatalytic method for enhancing arsenic methylation and volatilization of Clostridium and use thereof.

### BACKGROUND

Arsenic is a toxic metal element widely existing in the environment, primarily existing in the forms of inorganic trivalent and pentavalent arsenic. In addition to the naturally high arsenic levels in certain regions, human activities including fossil fuel development, ore smelting, pesticide use, etc. can also increase arsenic level in the environment, thereby increasing health risks associated with human exposure to arsenic. Microorganisms with arsenic-methylating function can convert inorganic trivalent arsenic into methyl arsenic with low toxicity and volatile arsenic gases, thereby reducing the concentration of available arsenic and helping to remove the arsenic from water bodies. Therefore, utilizing microbial arsenic methylation can provide an environmentally friendly and sustainable remediation technique for arsenic contaminated water bodies. As a detoxification mechanism of microorganisms, arsenic methylation and volatilization are catalyzed by the intracellular S-adenosylmethionine (SAM) methyltransferase ArsM, wherein the synthesis of the methyl donor SAM requires ATP consumption to drive the process. The efficiency of arsenic methylation is affected by both intracellular arsenic transformation processes (e.g. cellular arsenic efflux) and external environmental factors (e.g. redox potential and concentration of organic matter), which limit the effectiveness of arsenic volatilization in arsenic microbial remediation technique. Therefore, there is an urgent need to develop effective means to enhance arsenic volatilization in arsenic-contaminated groundwater and soil environments.

The Chinese Patent Application No. CN114480230A provides a strain of Clostridium sporogenes with the function of simultaneous anaerobic arsenic methylation and hydrogen production through fermentation. However, this strain has certain deficiencies in arsenic methylation and volatilization. Therefore, the present application is hereby provided.

### SUMMARY

An objective of a first aspect of the present disclosure is to provide use of resazurin in enhancing arsenic methylation capability of bacteria.

An objective of a second aspect of the present disclosure is to provide a method.

An objective of a third aspect of the present disclosure is to provide a method.

An objective of a fourth aspect of the present disclosure is to provide a reagent that is capable of enhancing the functions of arsenic methylation and volatilization of bacteria.

In order to achieve the above objectives, the technical solution adopted by the present disclosure is as follows.

The first aspect of the present disclosure provides use of resazurin in any one of (1) to (12):
(1) enhancing arsenic methylation capability of bacteria;
(2) preparing a product that enhances the arsenic methylation capability of bacteria;
(3) enhancing arsenic volatilization performance of bacteria;
(4) preparing a product that enhances the arsenic volatilization performance of bacteria;
(5) enhancing adsorption of arsenic by bacteria;
(6) preparing a product that enhances the adsorption of arsenic by bacteria;
(7) promoting growth of bacteria;
(8) preparing a product that promotes the growth of bacteria;
(9) inhibiting hydrogen production activity of bacteria;
(10) preparing a product that inhibits the hydrogen production activity of bacteria;
(11) inhibiting production of fermentation metabolites by bacteria; and
(12) preparing a product that inhibits the production of fermentation metabolites by bacteria; the fermentation metabolites comprise acetic acid and/or lactic acid.

In some embodiments of the present disclosure, the bacteria comprise at least one of Clostridium sporogenes, Bacillus spp., Shewanella spp., bacteria belonging to the family Chitinophagaceae, Chitinophaga spp., and Achromobacter xylosoxidans.

In some preferred embodiments of the present disclosure, the bacteria are Clostridium sporogenes, Bacillus spp. or combinations thereof.

In some preferred embodiments of the present disclosure, the bacteria are Clostridium sporogenes (deposited under accession number GDMCC No: 62212), Bacillus spp. (deposited under accession number GDMCC No: 62092) or combinations thereof.

In some embodiments of the present disclosure, the arsenic is trivalent arsenic.

In some embodiments of the present disclosure, the arsenic comprises arsenous acid and/or arsenites or combinations thereof.

In some embodiments of the present disclosure, the methylation of (1) to (2) comprises mono-methylation, di-methylation, and/or tri-methylation.

The second aspect of the present disclosure provides a reagent or kit comprising resazurin, glucose, and tryptone.

In some embodiments of the present disclosure, when the reagent or kit is used, a reaction system contains 1-600 μM of resazurin.

In some preferred embodiments of the present disclosure, when the reagent or kit is used, the reaction system contains 50-500 μM of resazurin.

In some embodiments of the present disclosure, when the reagent or kit is used, the reaction system contains 5-20 mM glucose and 0.5-3 g/L tryptone.

In some embodiments of the present disclosure, when the reagent or kit is used, the reaction system contains 7-15 mM glucose and 0.5-2 g/L tryptone.

In some embodiments of the present disclosure, when the reagent or kit is used, the reaction system contains 10-15 mM glucose and 1-2 g/L tryptone.

The third aspect of the present disclosure provides a method comprising a step of placing the resazurin or the reagent or kit of the second aspect of the present disclosure in an environment containing arsenic and bacteria;
wherein the method comprises any one of (a1) to (a3):
(a1) a method for enhancing arsenic methylation capability of the bacteria;
(a2) a method for enhancing arsenic volatilization performance of the bacteria; and
(a3) a method for enhancing adsorption of arsenic by the bacteria.

In some embodiments of the present disclosure, the methylation comprises mono-methylation, di-methylation, and/or tri-methylation.

In some embodiments of the present disclosure, the arsenic is inorganic trivalent arsenic.

In some embodiments of the present disclosure, the arsenic comprises arsenous acid and/or arsenites.

In some embodiments of the present disclosure, the environment is a culture medium, soil, or water body.

In some embodiments of the present disclosure, a concentration range of the inorganic trivalent arsenic in the environment or water body is 0-200 μmol/L.

In some embodiments of the present disclosure, the culture medium comprises nutrient broth supplemented with acetate and fumarate (NBAF) culture medium.

In some embodiments of the present disclosure, a carbon-nitrogen source in the culture medium comprises at least one of glucose, tryptone, and methionine.

In some embodiments of the present disclosure, the carbon-nitrogen sources in the culture medium are glucose and tryptone.

In some embodiments of the present disclosure, the bacteria comprise at least one of Clostridium sporogenes, Bacillus spp., Shewanella spp., bacteria belonging to the family Chitinophagaceae, Chitinophaga spp., and Achromobacter xylosoxidans.

In some preferred embodiments of the present disclosure, the bacteria are Clostridium sporogenes, Bacillus spp. or combinations thereof.

In some preferred embodiments of the present disclosure, the bacteria are Clostridium sporogenes (deposited under accession number GDMCC No: 62212), Bacillus spp. (deposited under accession number GDMCC No: 62092) or combinations thereof.

The fourth aspect of the present disclosure provides a method comprising a step of treating bacteria by using the resazurin or the reagent or kit of the second aspect of the present disclosure;
wherein the method comprises any one of (b1) to (b3):
(b1) a method for promoting growth of bacteria;
(b2) a method for inhibiting hydrogen production activity of bacteria; and
(b3) a method for inhibiting production of fermentation metabolites by bacteria.

In some embodiments of the present disclosure, the fermentation metabolites comprise acetic acid and/or lactic acid.

In some embodiments of the present disclosure, the bacteria comprise at least one of Clostridium sporogenes, Bacillus spp., Shewanella spp., bacteria belonging to the family Chitinophagaceae, Chitinophaga spp., and Achromobacter xylosoxidans.

In some preferred embodiments of the present disclosure, the bacteria are Clostridium sporogenes, Bacillus spp. or combinations thereof.

In some preferred embodiments of the present disclosure, the bacteria are Clostridium sporogenes (deposited under accession number GDMCC No: 62212), Bacillus spp. (deposited under accession number GDMCC No: 62092) or combinations thereof.

The beneficial effects of the present disclosure are as follows.

The present disclosure proposes for the first time that resazurin can enhance the functions of bacteria (such as Clostridium sporogenes) in arsenic methylation, volatilization, and adsorption. Experiments have proven that exogenous addition of resazurin can significantly enhance the methylation and volatilization of inorganic trivalent arsenic by Clostridium sporogenes.

Specifically, 50-500 μM of resazurin significantly promotes the production of water-soluble methyl arsenic by Clostridium sporogenes compared to the blank treatment, with the production increases with increasing concentrations of resazurin, wherein when treated with 500 μM of resazurin, compared to the blank treatment, the concentrations of water-soluble dimethyl arsenic and trimethyl arsenic are increased by 2.3-fold and 2.7-fold, respectively. The dimethyl arsenic accounts for 95.6%-96.6% of the total methyl arsenic. The production of volatile arsenic (primarily dimethyl arsenic hydride, accounting for 90.8%-94.9% of the total volatile arsenic) also shows a significant increase trend with the addition of resazurin, with no significant difference in volatile arsenic between the 100 μM and 300 μM treatments with resazurin, while the 500 μM treatment shows a 3.2-fold increase in volatile arsenic compared to the blank treatment. Compared to the blank treatment (0.47 μg/L), the presence of resazurin significantly increases the concentration of cellular adsorbed arsenic by 1.4-2.1 folds, wherein there is no significant difference in cellular adsorbed methyl arsenic between the 100 μM and 300 μM treatments with resazurin, with the dimethyl arsenic accounts for 67.7%-83.4% of the total adsorbed methyl arsenic in all treatments.

50-500 μM of resazurin significantly promotes the growth of Clostridium sporogenes by 11.7%-18.3%. For the fermentation metabolites, the presence of resazurin reduces the production of lactic acid, with the production of lactic acid decreases with increasing concentrations of resazurin. The lowest production of lactic acid is observed at the resazurin concentration of 500 μM, which is decreased by 52.5% than the blank treatment. For the fermentation product acetic acid, the production of acetic acid in the resazurin treatment groups decreases with increasing resazurin concentrations, with a reduction of 21.1%-38.8% compared to the group without resazurin treatment, wherein the lowest production of acetic acid is observed under 500 μM resazurin treatment. The cumulative production of hydrogen by resazurin treatment decreases with increasing resazurin concentrations, with a reduction of 5.3%-25% compared to the treatment without resazurin (12.3 mM), wherein the lowest cumulative production of hydrogen (9.2 mM) is observed at the condition of 500 μM resazurin.

It can be seen that Clostridium sporogenes can accelerate growth and arsenic volatilization under the condition of exogenous addition of resazurin, demonstrating potential applications in carbon-neutral arsenic pollution remediation.

The method provided by the present disclosure can efficiently enhance the arsenic methylation and volatilization rate of Clostridium, providing an effective means for the remediation of arsenic-contaminated soil and water bodies, while also featuring characteristics of easy-to-operate and cost-effective.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows the results of the effect of carbon-nitrogen organic substrate components in the culture medium on the production of water-soluble methyl arsenic by C. sporogenes LHA6;

FIGS. 2a-2d show the results of the effect of different concentrations of resazurin (RZ) on the arsenic methylation and volatilization of C. sporogenes LHA6; wherein FIG. 2a shows the change in concentrations of water-soluble dimethyl arsenic over cultivation time; FIG. 2b shows the change in concentrations of water-soluble trimethyl arsenic over cultivation time; FIG. 2c shows the compositions and concentrations of volatile methyl arsenic after 72 hours of cultivation; and FIG. 2d shows the compositions and concentrations of cellular adsorbed methyl arsenic after 72 hours of cultivation; and

FIGS. 3a-3b show the results of the effect of different concentrations of resazurin (RZ) on the growth and production of fermentation products of C. sporogenes LHA6; wherein FIG. 3a shows the protein content and concentrations of short-chain fatty acids acetic acid and lactic acid after 72 hours of cultivation; and FIG. 3b shows the change in concentrations of headspace hydrogen over cultivation time.

### DETAILED DESCRIPTION

The following specific embodiments are provided to further illustrate the content of the present disclosure in detail.

It should be understood that these embodiments are provided only for the purpose of illustrating the present disclosure and are not intended to limit the scope of the present disclosure.

In order to clarify the purpose, the technical solution, and the advantages of the embodiments of the present disclosure, a clear and complete description of the technical solution in the embodiments of the present disclosure will be provided below. For the conditions not specified in the embodiments, conventional conditions or conditions recommended by the manufacturer shall be applicable. The used reagents or instruments without specifying the manufacturers are conventional products that can be purchased commercially.

The following provides a further detailed description of the features and performance of the present disclosure in conjunction with the embodiments.

### Example 1. Differences in Arsenic Conversion by Arsenic-Methylating Functional Clostridium LHA6 Under Different Carbon-Nitrogen Source Conditions

Clostridium sporogenes LHA6 (deposited under accession number GDMCC No: 62212 on Jan. 14, 2022, at Guangdong Microbial Culture Collection Center located at Building 59, No. 100 Xianlie Central road, Yuexiu District, Guangzhou, Guangdong, China, and disclosed in Patent CN114480230A).

Firstly, in order to obtain seed culture of strain in the logarithmic growth phase, LHA6 was inoculated at a concentration of 5% (v/v) into ¼ TSB medium containing 30 mM piperazine-1,4-diethanesulfonic acid (PIPES) buffer (pH=7.0), with a culture volume of 50 mL in a 120 mL vial. High-purity nitrogen gas was introduced for 30 minutes before sealing to form an anaerobic environment. The culture was then incubated under static cultivation in the dark at 30° C. for 30 hours to obtain the seed culture of strain in the logarithmic growth phase, with a cell density of 0.8×10⁸ cells/mL to 1.6×10⁸ cells/mL. The seed culture obtained was centrifuged at 5000×g for 10 minutes, bacteria were washed three times with physiological saline, and then resuspended in modified NBAF culture medium. The resuspended seed culture was inoculated at a concentration of 5% (v/v) into modified NBAF culture medium containing arsenic, with a sodium arsenite concentration of 30 μM. The components of the modified NBAF culture medium containing arsenic comprise CaCl₂·2H₂O 0.04 g/L, MgSO₄·7H₂O 0.1 g/L, NaHCO₃1.8 g/L, Na₂CO₃ 0.4273 g/L, Na₂SeO₄ 0.001 mM, 100×NB Salts 10 mL/L, and NB Mineral Elixir 10 mL/L. The composition of the 100×NB Salts is as follows: KH₂PO₄ 42 g/L, K₂HPO₄ 22 g/L, NH₄Cl 20 g/L, KCl 38 g/L, and NaCl 36 g/L. The composition of the NB Mineral Elixir is as follows: Nitrilotriacetic acid 2.14 g/L, MnCl₂·4H₂O 0.1 g/L, FeSO₄·7H₂O 0.3 g/L, CoCl₂·6H₂O 0.17 g/L, ZnSO⁴·7H₂O 0.2 g/L, CuCl₂·2H₂O 0.03 g/L, AlK(SO₄)₂·12H₂O 0.005 g/L, H₃BO₃ 0.005 g/L, Na₂MnO₄·2H₂O 0.09 g/L, NiSO₄·6H₂O 0.11 g/L, and Na₂WO₄·2H₂O 0.02 g/L.

In order to determine the optimal carbon-nitrogen components of the culture medium for arsenic methylation of the strain, different treatments with organic substrate were set up, including methionine (10 mM), methionine (10 mM)+tryptone (1 g/L), glucose (10 mM), and glucose (10 mM)+tryptone (1 g/L). The volume of the culture system was 50 mL in a 120 mL vial, and the pH of the culture system was adjusted to 7.0. A CO₂:N₂ (20:80) gas mixture was introduced for 30 minutes before sealing, and the culture was then incubated under static cultivation in the dark at 30° C. for 72 hours. After the culture was completed, liquid sample was collected and filtered through a 0.22 μm filter. The concentrations of soluble trimethyl arsenic, dimethyl arsenic, and monomethyl arsenic in the solution were determined using high-performance liquid chromatography inductively coupled plasma mass spectrometry (HPLC-ICP-MS, PerkinElm).

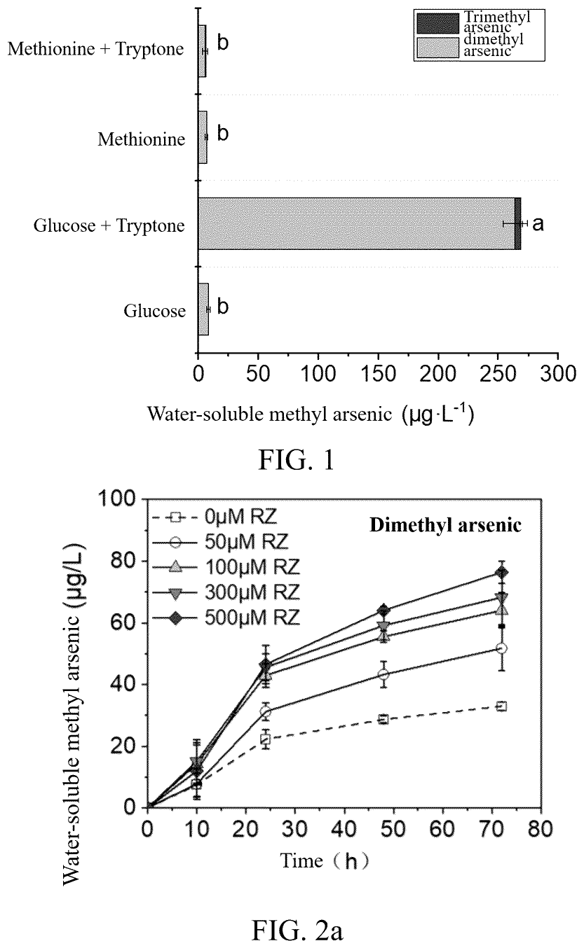

The results of production pf methyl arsenic by LHA6 under different carbon-nitrogen organic substrate treatments were shown in FIG. 1. The amount of dimethyl arsenic produced under glucose treatment was 8.4 μg/L, and the amount of dimethyl arsenic produced under glucose+tryptone composite substrate treatment was increased by 31.4-fold compared to that under glucose treatment. The production of trimethyl arsenic showed a similar trend, with the amount of trimethyl arsenic produced under the glucose treatment being 0.3 μg/L, which was increased to 5.1 μg/L under the composite substrate treatment. The amounts of dimethyl arsenic and trimethyl arsenic produced under treatment using methionine as the sole substrate showed no significant difference compared to treatments using methionine+tryptone or glucose alone. These results indicated that the composite substrate of tryptone with carbohydrate substrate (glucose) is more favorable for the arsenic methylation process of the LH16 strain compared to the composite substrate with amino acid (methionine) substrate. Therefore, the composite substrate of glucose and tryptone was recommended as the preferred substrate for the arsenic methylation reaction of LHA6.

### Example 2. Promoting Effect of Resazurin on Arsenic Methylation and Volatilization of Clostridium LHA6

After obtaining the seed culture of strain in the logarithmic growth phase using the method as described in Example 1, it was inoculated at a concentration of 5% (v/v) into the modified NBAF culture medium containing arsenic, with a sodium arsenite concentration of 30 μM and glucose (10 mM)+tryptone (1 g/L) served as the organic substrate. In order to determine the optimal resazurin (ZR) concentration for arsenic methylation and volatilization in the culture system, concentration gradients were set up for resazurin, including 0 μM, 50 μM, 100 μM, 300 μM, and 500 μM. The volume of the culture system was 50 mL in a 120 mL vial, and the pH of the culture system was adjusted to 7.0. A CO₂:N₂ (20:80) gas mixture was introduced for 30 minutes before sealing, and the culture was then incubated under static cultivation in the dark at 30° C. for 72 hours.

Liquid samples were collected at various time points during the cultivation process, and the bacteria and headspace arsenic gas were collected at the end of 72 hours of the cultivation, for testing cellular adsorbed arsenic and volatile arsenic. All the culture broth was centrifuged at 5000×g for 10 minutes, the supernatant was discarded, and the bacteria were washed three times with PIPES buffer. A liquid obtained by digesting with 0.5% nitric acid for 1 hour was used for determining cellular adsorbed methyl arsenic. Chemical capture of volatile arsenic compounds was achieved by their reaction with solid oxidant AgNO₃. High-purity nitrogen gas (100 kPa) filtered through a 0.22 μm syringe filter was blown into the headspace of the anaerobic tube for 10 minutes. The outlet of nitrogen gas was connected to a 1 mL syringe containing high-purity silica gel particles (Sigma, Davisil Grade 636, pore size 60 Å, 35-60 mesh particle size) soaked in 10% AgNO₃. The silica gel particles after capturing arsenic were refluxed and digested in 2 mL of 0.5% (v/v) HNO₃ at 90° C. for 1 hour to recover volatile arsenic. 5 μL of H₂O₂ (3%) was added to 0.5 mL of the recovered solution to oxidize CH₃AsH₂, (CH₃)₂AsH, and (CH₃)₃As to monomethyl arsenic, dimethyl arsenic, and trimethyl arsenic, respectively. All liquid samples were filtered through a 0.22 μm filter and the concentrations of soluble trimethyl arsenic, dimethyl arsenic, and monomethyl arsenic were determined using the aforementioned high-performance liquid chromatography inductively coupled plasma mass spectrometry.

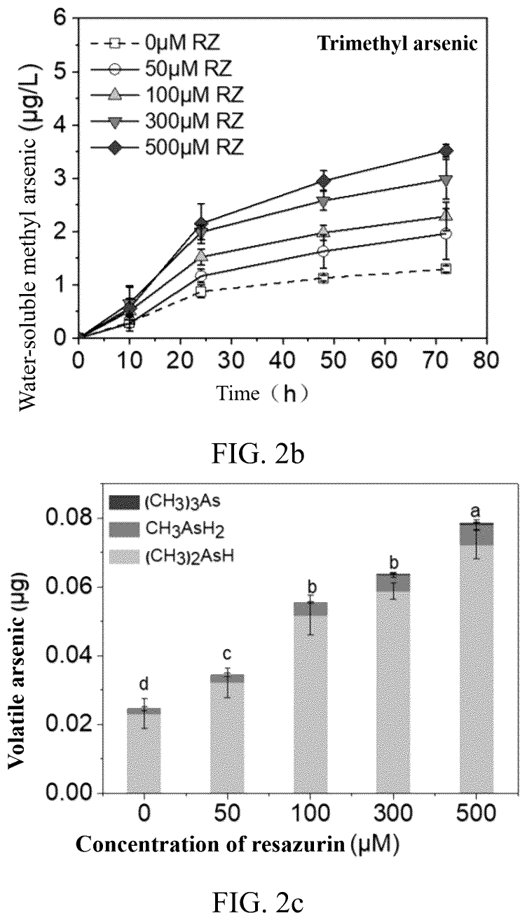

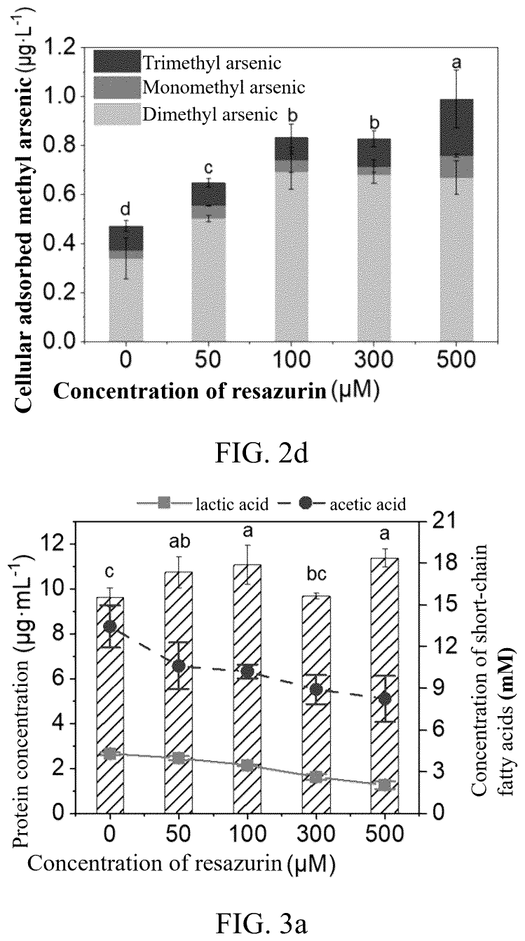

Quantitative analysis of the soluble and volatile methyl arsenic products of LHA6 strain treated with different concentrations of resazurin revealed that methyl arsenic continuously accumulated in LHA6 during the 72-hour cultivation period, with 33 μg/L of water-soluble dimethyl arsenic and 1.3 μg/L of water-soluble trimethyl arsenic produced at 72 h (FIG. 2a). In the resazurin treatment groups, 50-500 μM of resazurin significantly promoted the production of water-soluble methyl arsenic in LHA6 compared to the blank treatment, with the production increased with increasing concentrations of resazurin, wherein compared to the blank treatment, the concentrations of water-soluble dimethyl arsenic and trimethyl arsenic in the 500 μM treatment group were increased by 2.3-fold and 2.7-fold, respectively (FIG. 2b). The dimethyl arsenic accounted for 95.6%-96.6% of the total methyl arsenic in all treatment groups. The volatile arsenic production of LHA6 strain in the blank treatment was 0.025 μg. The production of volatile arsenic also showed a significant increase trend with the addition of resazurin, with no significant difference in volatile arsenic between the 100 μM and 300 μM treatments with resazurin, while the 500 μM treatment showed a 3.2-fold increase in volatile arsenic compared to the blank treatment (FIG. 2c). In all treatments, the form of volatile methyl arsenic was primarily dimethyl arsenic hydride, accounting for 90.8%-94.9% of the total volatile arsenic. Compared to the blank treatment (0.47 μg/L), the presence of resazurin significantly increased the concentration of cellular adsorbed arsenic by 1.4-2.1 folds, wherein there was no significant difference in cellular adsorbed methyl arsenic between the 100 μM and 300 μM treatments with resazurin, with the dimethyl arsenic accounted for 67.7%-83.4% of the total adsorbed methyl arsenic in all treatments (FIG. 2d). These results indicated that exogenous resazurin could increase efficiency of arsenic methylation and volatilization by LHA6.

### Example 3. Effect of Resazurin on Growth and Change of Fermentation Products of Arsenic-Methylating Clostridium LHA6

The present example was used to verify the growth and fermentation products of arsenic-methylating Clostridium LHA6 under treatment conditions of different resazurin concentrations in Example 2, specifically as follows.

After cultivation, the bacteria were collected, washed three times with PIPES buffer, and then resuspended to 5 mL. 0.3 mL of the resuspended cell suspension was taken, added to 1 mL of 0.2 M NaOH solution, and incubated at 100° C. with shaking at 120 rpm for 30 minutes to obtain cell lysate. Protein concentration in the lysate was determined using an improved Bradford protein concentration assay kit (Sangon, China) to characterize cell biomass. Organic acids were detected by high-performance liquid chromatography with a UV detector (Agilent 1260, USA) using a C18 column (4.6 mm×250 mm) at a column temperature of 30° C. The mobile phase was 0.1 M NaH₂PO₄·2H₂O adjusted to pH 3.1, with a flow rate of 1 mL/min.

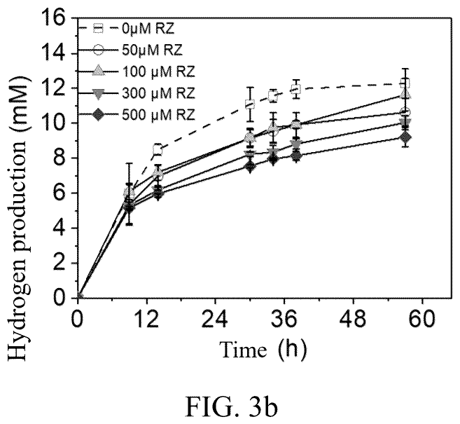

50-100 μM and 500 μM of resazurin significantly promoted the growth of LHA6 by 11.7%-18.3% compared to the blank treatment (FIG. 3a). Resazurin promoted cell growth at low concentrations (50-100 μM), which might be related to the enhanced extracellular electron transfer flux of cells by AQDS and resazurin. For the fermentation metabolites, the presence of resazurin reduced the production of lactic acid, with the production of lactic acid decreased with increasing concentrations of resazurin. The lowest production of lactic acid was observed at the resazurin concentration of 500 μM, which was decreased by 52.5% than the blank treatment. For the fermentation product acetic acid, the production of acetic acid in the resazurin treatment groups decreased with increasing resazurin concentrations, with a reduction of 21.1%-38.8% compared to the group without resazurin treatment, wherein the lowest production of acetic acid was observed under 500 μM resazurin treatment (FIG. 3a). The cumulative production of hydrogen by resazurin treatment decreased with increasing resazurin concentrations, with a reduction of 5.3%-25% compared to the treatment without resazurin (12.3 mM), wherein the lowest cumulative production of hydrogen was observed at the condition of 500 μM resazurin (9.2 mM) (FIG. 3b). Further standardization of hydrogen production based on protein concentration was used to reflect the hydrogen production capability per unit mass of the strain. The results showed that in the presence of resazurin, the hydrogen production of LHA6 strain (0.81-1.05 mmol/mg) was decreased by 17.6%-36.6% compared to the treatment without resazurin, indicating that resazurin inhibited the hydrogen production activity of the strain.

The above results indicated that resazurin had inhibitory effects on production of hydrogen, fermentation metabolites acetic acid and lactic acid of the bacterial strain. Due to the higher redox potential of resazurin (−51 mV) than that of hydrogen (−410 mV), this potential difference suggested that the presence of resazurin may help to enhance extracellular electron transfer flux of Clostridium. In addition, resazurin was a small-molecule lipophilic electron shuttle that could directly enter the periplasm and interact with periplasmic and inner membrane proteins to affect electron distribution, thereby potentially affecting intracellular carbon metabolic pathways. Therefore, by modulating the intracellular carbon metabolic pathways, resazurin inhibited fermentation metabolic activity while enhancing arsenic methylation reaction and the production of volatile methyl arsenic.

The embodiments of the present disclosure have been described in detail above in conjunction with the accompanying drawings, However, the present disclosure is not limited to the above embodiments. Various changes can be made within the scope of knowledge possessed by those ordinary skilled in the art without departing from the purpose of the present disclosure. In addition, the embodiments of the present disclosure and the features in the embodiments may be combined with each other without conflict.

What is claimed is:

1. A method, comprising a step of placing resazurin, a reagent comprising resazurin, or a kit comprising resazurin in an environment containing arsenic and bacteria;
wherein the method comprises any one of (a1) to (a3):
(a1) a method for enhancing arsenic methylation capability of bacteria under an anaerobic condition;
(a2) a method for enhancing arsenic volatilization performance of bacteria under an anaerobic condition; and
(a3) a method for enhancing adsorption of arsenic by bacteria under an anaerobic condition;
the bacteria are Clostridium sporogenes with an accession number GDMCC No: 62212;
the reagent comprising resazurin further comprises glucose, and tryptone; and the kit comprising resazurin further comprises glucose, and tryptone;
in the method (a1) and method (a2), the resazurin has an effective concentration of 50 μM to 500 μM; and
the methylation comprises mono-methylation, di-methylation, and/or tri-methylation.

2. The method according to claim 1, wherein the arsenic is inorganic trivalent arsenic.

3. The method according to claim 2, wherein the arsenic comprises arsenous acid and/or arsenites.

4. The method according to claim 2, wherein the environment is a culture medium, soil, or water body.

5. The method according to claim 4, a concentration range of the inorganic trivalent arsenic in the environment or the water body is 0-200 μmol/L.

6. The method according to claim 4, wherein the culture medium comprises a nutrient broth supplemented with acetate and fumarate (NBAF) culture medium.

7. The method according to claim 6, wherein a carbon-nitrogen source in the culture medium comprises at least one of glucose, tryptone, and methionine.

8. The method according to claim 6, wherein the carbon-nitrogen source in the culture medium are glucose and tryptone.

9. A method, comprising a step of treating bacteria by using resazurin, a reagent comprising resazurin, or a kit comprising resazurin;
wherein the method comprises any one of (b1) to (b3):
(b1) a method for promoting growth of bacteria under an anaerobic condition;
(b2) a method for inhibiting hydrogen production activity of bacteria under an anaerobic condition; and
(b3) a method for inhibiting production of fermentation metabolites by bacteria under an anaerobic condition;
the bacteria are Clostridium sporogenes with an accession number GDMCC No: 62212;
the reagent comprising resazurin further comprises glucose, and tryptone; and the kit comprising resazurin further comprises glucose, and tryptone;
the fermentation metabolites are acetic acid and/or lactic acid.
